(19) United States
(12) Patent Application Publication
(10) Pub. No.: US 2024/0081896 A1
(43) Pub. Date: Mar. 14, 2024

(54) METHODS AND APPARATUS FOR PERFORMING MEDICAL PROCEDURES USING RADIOFREQUENCY ENERGY

(71) Applicant: ELECTROWIRE CORPORATION, Lexington, SC (US)

(72) Inventor: Robert Leonardi, Lexington, SC (US)

(21) Appl. No.: 18/243,927

(22) Filed: Sep. 8, 2023

Related U.S. Application Data
(60) Provisional application No. 63/405,060, filed on Sep. 9, 2022.

(51) Int. Cl. A61B 18/14

(52) U.S. Cl. CPC A61B 18/1482; A61B 2017/00477; A61B 2018/144

(57) ABSTRACT

An apparatus for performing a medical procedure including an electrosurgical unit and an assembly including an elongated flexible conductive element, an interventional wire, a coupler, and an activator unit. The electrosurgical unit is configured to generate radiofrequency energy. The elongated flexible conductive element includes a proximal end connected to the electrosurgical unit, and a distal end. The interventional wire is configured to deliver radiofrequency energy. The coupler electrically couples the elongated flexible conductive element to the interventional wire. The activator unit is electrically coupled to the elongated flexible conductive element and is configured to selectively activate the delivery of radiofrequency energy from the electrosurgical unit to the interventional wire.

### CROSS-REFERENCE TO RELATED APPLICATION

This application claims the benefit of U.S. Provisional Patent Application Ser. No. 63/405,060, filed Sep. 9, 2022 (pending), the disclosure of which is incorporated by reference herein in its entirety.

### TECHNICAL FIELD

The present disclosure generally relates to the delivery of radiofrequency (RF) energy from a supplier of RF energy to the tip of an interventional wire, via a conductor, to perform medical procedures.

### BACKGROUND

Advances in cardiology have caused an increase in transseptal procedures and accordingly, an increase in the required precision of transseptal procedures. Transseptal procedures known as transseptal punctures are critical to gain access to the heart. More specifically, transseptal punctures allow medical professionals to gain access to the left atrium of the heart. Access to the left atrium of the heart is obtained through the right atrium of the heart after the medical professional has gained access to the heart, either from the jugular vein through the superior vena cava, or from the femoral vein through the inferior vena cava.

Conventional transseptal procedures utilize needles to make the puncture across the septum from the right atrium of the heart to the left atrium of the heart to allow the medical professional to gain access to the left atrium. Needles, though, are not the most ideal manner of making this type of puncture. Needles tend to slide superiorly when advanced with the intent to puncture, leading to off-target punctures and further, needles cannot be utilized to deliver necessary medical devices to the left atrium, so additional steps are required when these medical devices need to access the left atrium.

Radiofrequency-assisted devices are also being used to puncture through septal tissue within the heart and/or to gain access the left atrium of the heart. One such technology uses a radiofrequency energy-based guidewire to pierce the septum located between the right atrium of the heart and the left atrium of the heart. But, radiofrequency-assisted guidewire devices of this type can be expensive and, therefore, this technology may be limited in use. For example, existing radiofrequency-assisted devices used for septal heart tissue puncturing may require expensive electrosurgical, RF energy generating units, as opposed to the more conventional, less costly electrosurgical, RF energy generating units used for many surgical procedures other than septal puncturing of heart tissue.

For these and other reasons, there is a need in this field for radiofrequency-assisted devices, systems and methods that may be used to perform medical procedures, such as transseptal procedures, with lower cost and higher efficiency, thereby giving medical professionals and their patients better choices at lower cost, while still achieving the desired accuracy and ease of use.

### SUMMARY

Generally, an apparatus for performing a medical procedure is provided. The apparatus includes an electrosurgical unit and an assembly including an elongated flexible conductive element, an interventional wire, a coupler, and an activator unit. The electrosurgical unit is configured to generate radiofrequency energy. The elongated flexible conductive element includes a proximal end connected to the electrosurgical unit, and a distal end. The interventional wire is configured to deliver radiofrequency energy. The coupler electrically couples the distal end of the elongated flexible conductive element to the interventional wire. The activator unit is electrically coupled to the elongated flexible conductive element and is configured to selectively activate the delivery of radiofrequency energy from the electrosurgical unit to the interventional wire.

In some embodiments, the interventional wire may be an insulated wire with a proximal end portion and a distal end portion. The proximal end portion may be configured for coupling the interventional wire to the coupler. An uninsulated tip portion may be located at the distal end portion of the insulated wire and configured to deliver radiofrequency energy. The proximal end portion of the interventional wire may include an uninsulated proximal end portion. The elongated flexible conductive element may be a cable. The coupler may include a collet configured to electrically couple the elongated flexible conductive element and the interventional wire. The elongated flexible conductive element may be coupled to a proximal end of the collet and the interventional wire may be coupled to a distal end of the collet. The coupler may also include a housing and a cap. The housing may at least partially contain the collet. The cap may be coupled to a distal end of the housing. The interventional wire may be coupled to the collet with the cap.

In alternate or additional aspects, the interventional wire may be removably coupled to the coupler. Tightening the cap may compress or tighten the distal end of the collet on the interventional wire thereby coupling the interventional wire to the collet. Loosening the cap may allow the distal end of the collet to loosen or expand allowing the interventional wire to be removed from the collet. The interventional wire may serve as a guidewire for the delivery of a medical device. The activator unit may include a switch element that is configured to selectively supply radiofrequency energy from the electrosurgical unit through the elongated flexible conductive element to the interventional wire when the switch element is activated. The activator unit may be spatially separated from the coupler by a portion of the elongated flexible conductive element. The activator unit may be spatially separated from the proximal end of the elongated flexible conductive element by a segment of the elongated flexible conductive element. The apparatus may include an electrosurgical unit connector coupled to the proximal end of the elongated flexible conductive element and configured to connect the elongated flexible conductive element to the electrosurgical unit. The elongated flexible conductive element may be releasably connected to the electrosurgical unit. The apparatus may include an electroanatomical mapping connector for connecting the apparatus to an electroanatomical mapping system.

An alternative apparatus for performing a medical procedure is provided. The alternative apparatus includes an elongated flexible conductive element, an interventional wire, a coupler, and an activator unit. The elongated flexible conductive element includes a proximal end configured to connect to an electrosurgical unit for generating radiofrequency energy, and a distal end. The interventional wire is configured to deliver radiofrequency energy. The coupler electrically couples the distal end of the elongated flexible conductive element to the interventional wire. The activator unit is electrically coupled to the elongated flexible conductive element and is configured to selectively activate the delivery of radiofrequency energy from an electrosurgical unit to the interventional wire.

In alternative embodiments, the coupler includes a collet configured to electrically couple the elongated flexible conductive element and the interventional wire. The elongated flexible conductive element may be coupled to a proximal end of the collet. The interventional wire may be coupled to a distal end of the collet. The coupler may include a housing and a cap. The housing may at least partially contain the collet. The cap may be coupled to a distal end of the housing. The interventional wire may be coupled to the collet with the cap. The interventional wire may be removably coupled to the coupler. Tightening the cap may compress or tighten the distal end of the collet on the interventional wire thereby coupling the interventional wire to the collet. Loosening the cap may allow the distal end of the collet to loosen or expand allowing the interventional wire to be removed from the collet.

In alternative or additional aspects, the interventional wire may be removably coupled to the coupler. The interventional wire may serve as a guidewire for the delivery of a medical device. The activator unit may include a switch element that is configured to selectively supply the radiofrequency energy from the electrosurgical unit through the elongated flexible conductive element to the interventional wire when the switch element is activated. The activator unit may be spatially separated from the coupler by a portion of the elongated flexible conductive element. The activator unit may be spatially separated from the proximal end of the elongated flexible conductive element by a segment of the elongated flexible conductive element. The apparatus may include an electroanatomical mapping connector for connecting the apparatus to an electroanatomical mapping system. The proximal end of the elongated flexible conductive element may include an electrosurgical unit connector configured to couple the elongated flexible conductive element to an electrosurgical unit. The electrosurgical unit connector may be configured to couple the elongated flexible conductive element to any one of a plurality of electrosurgical units.

In another embodiment, an apparatus for performing a medical procedure includes an electrosurgical unit and an assembly including an elongated flexible conductive element, an interventional wire, a coupler, and an activator unit. The electrosurgical unit is configured to generate radiofrequency energy. The elongated flexible conductive element includes a proximal end connected to the electrosurgical unit, and a distal end. The interventional wire includes an insulated wire with an uninsulated tip portion located at a distal end portion of the interventional wire and configured to deliver radiofrequency energy. The coupler is configured to removably couple a proximal end portion of the interventional wire, electrically and mechanically, to the distal end of the elongated flexible conductive element. The activator unit is electrically coupled to the elongated flexible conductive element and is configured to selectively activate the delivery of radiofrequency energy from the electrosurgical unit to the interventional wire.

In some embodiments, tightening the coupler may couple the interventional wire to the coupler. Loosening the coupler may allow the interventional wire to be removed from the coupler. The coupler may include a collet configured to electrically couple the elongated flexible conductive element and the interventional wire. The elongated flexible conductive element may be coupled to a proximal end of the collet. The interventional wire may be coupled to a distal end of the collet. In alternative embodiments, the coupler may include a housing at least partially containing the collet, and a cap coupled to a distal end of the housing. The interventional wire may be coupled to the collet with the cap. Tightening the cap may compress or tighten the distal end of the collet on the interventional wire thereby coupling the interventional wire to the collet. Loosening the cap may allow the distal end of the collet to loosen or expand allowing the interventional wire to be removed from the collet. The interventional wire may serve as a guidewire for the delivery of a medical device. The activator unit may include a switch element that is configured to selectively supply the radiofrequency energy from the electrosurgical unit through the elongated flexible conductive element to the interventional wire when the switch element is activated. The activator unit may be spatially separated from the coupler by a portion of the elongated flexible conductive element. The activator unit may be spatially separated from the proximal end of the elongated flexible conductive element by a segment of the elongated flexible conductive element. The apparatus may include an electrosurgical unit connector coupled to the proximal end of the elongated flexible conductive element and configured to connect the elongated flexible conductive element to the electrosurgical unit. The elongated flexible conductive element may be releasably connected to the electrosurgical unit. The apparatus may include an electroanatomical mapping connector for connecting the apparatus to an electroanatomical mapping system.

A method of performing a medical procedure is provided and includes generating radiofrequency energy using an electrosurgical unit and conducting the radiofrequency energy from the electrical surgical unit through an elongated flexible conductive element to an activator unit. The method also includes selectively activating the activator unit to selectively direct the radiofrequency energy to an interventional wire, and using the interventional wire to deliver the radiofrequency energy to a surgical site. The surgical site may be the heart and the medical procedure performed may be a transeptal puncture. The activator may be selectively activated by activating and deactivating a switch element. The method may include generating an electroanatomical map. The method may include guiding the interventional wire to a surgical site. The method may include guiding the interventional wire through an introducer sheath. The method may include guiding a medical device to a surgical site with the interventional wire.

A method of preparing an apparatus for a medical procedure is provided and includes mechanically and electrically coupling an elongated flexible conductive element to an electrosurgical unit for conducting radiofrequency energy generated by the electrosurgical unit. The method also includes mechanically and electrically coupling the elongated flexible conductive element to a coupler, and mechanically and electrically coupling an interventional wire to the coupler. The method may include coupling the electrosurgical unit to an electroanatomical mapping system. The method may include energizing the electrosurgical unit. The method may include guiding the interventional wire into an introducer sheath.

Any of the features and functions described herein may be applied to any of the disclosed embodiments or methods. Additional features and advantages of the inventive aspects disclosed herein will become more apparent upon review of the following detailed description taken together with accompanying drawings of the illustrative and exemplary embodiments.

### BRIEF DESCRIPTION OF THE DRAWINGS

FIG. 1 is a perspective view of an illustrative apparatus for delivering RF energy to tissue during a medical procedure.

FIG. 2 is an exploded view of a portion of a coupler that is a component of the apparatus of FIG. 1.

FIG. 3 is a sectional view of the coupler of FIGS. 1 and 2.

FIG. 4 is a perspective view of a pigtail loop formed by the interventional wire of the apparatus.

### DETAILED DESCRIPTION OF THE ILLUSTRATIVE EMBODIMENT

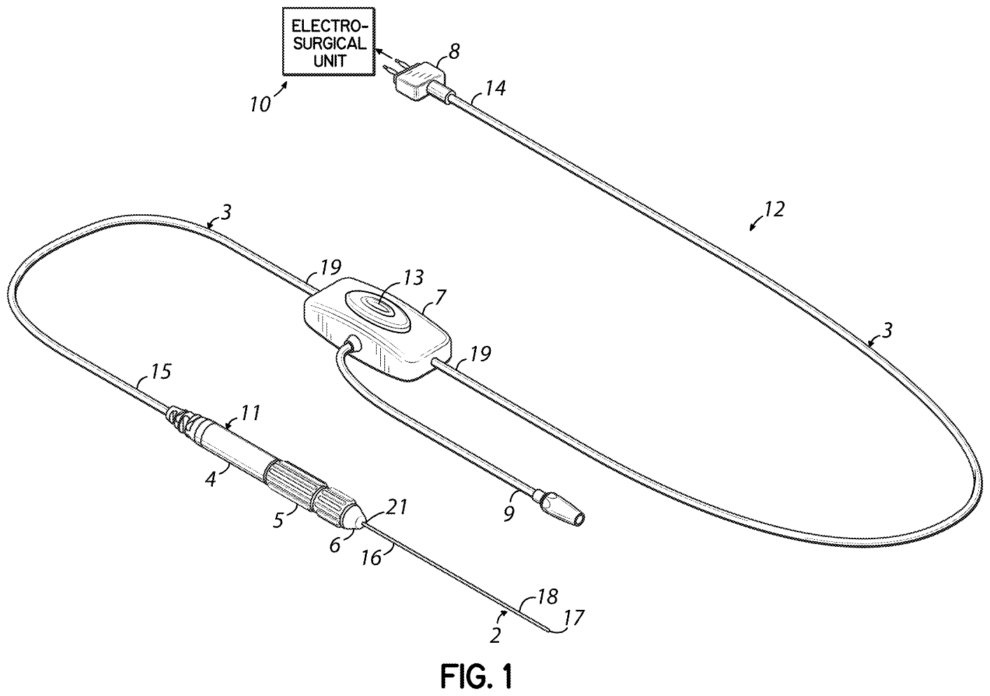

FIG. 1 is a perspective view of an illustrative apparatus for delivering RF energy to tissue during a medical procedure. In this illustrative embodiment, an interventional wire 2, an elongated flexible conductive element 3, an activator unit 7, an electrosurgical unit 10, and a coupler 11 form the apparatus 12 which is configured for performing a medical procedure. The elongated flexible conductive element 3 is an electrically insulated conductor, such as a wire, configured for transmitting electricity or RF energy. In some embodiments, the elongated flexible conductive element 3 may be a cable including an insulated wire or wires and having a protective casing. The elongated flexible conductive element 3 includes a proximal end 14 including an electrosurgical unit connector 8, and a distal end 15 coupled to the coupler 11. The electrosurgical unit connector 8, capable of attaching to conventional RF energy generating units for delivering RF energy, may releasably connect to the electrosurgical unit 10. Many commercially available electrosurgical units include a standardized receptacle, such as a monopolar accessory receptacle. The electrosurgical unit connector 8 may be configured to couple the apparatus 12 to any one of a plurality of electrosurgical units with standardized receptacles. Therefore, the subsystem or assembly comprising, for example, interventional wire 2, elongated flexible conductive element 3, activator unit 7, and coupler 11 may be physically coupled to one of several different standardized receptacles of an electrosurgical RF generating unit. In the illustrative embodiment the elongated flexible conductive element 3 has a mid-portion 19 that is connected to the activator unit 7. The activator unit 7 is situated at a location spatially separated from the proximal end 14 of the elongated flexible conductive element 3 and, therefore also spatially separated from the electrosurgical unit 10, by a segment of the elongated flexible conductive element 3. The activator unit 7 also may be spatially separated from the coupler 11 by another segment of the elongated flexible element 3, as shown in FIG. 1 or, optionally, the activator unit 7 may be integrated with or otherwise fixed to the coupler 11. In some embodiments, the activator unit 7 may be located at the proximal end 14 of the elongated flexible conductive element 3 adjacent to or otherwise fixed to the electrosurgical unit connector 8. The elongated flexible conductive element 3 may be constructed in multiple, discrete pieces or segments or the segments referred to herein may be portions of the same integral elongated flexible conductive element 3.

The apparatus 12 includes an electroanatomical mapping connector 9 that is capable of connecting to electroanatomical mapping systems. Procedural efficiency and safety may be improved with the use of electroanatomical mapping systems. Electroanatomical mapping systems are used by medical practitioners to improve awareness of the location of diagnostic and interventional devices in the human body during minimally invasive procedures, including transseptal puncture. Electroanatomical mapping systems have advantages over fluoroscopy including: visualization in three dimensions as opposed to two, reducing the use of ionizing radiation, and allowing real-time visualization of diagnostic and interventional devices during a procedure. The electroanatomical mapping connector 9 facilitates connection of the apparatus 12 to a mapping system “dongle,” an adapter that allows various mapping catheters from different manufacturers to connect to the electroanatomical mapping system. Once the apparatus 12 is connected to an electroanatomical mapping system, the tip 17 of the interventional wire 2 functions as the tip of a mapping catheter and may be visualized on a three-dimensional electroanatomical map. The location of the tip 17 of the interventional wire 2 will be evident in real time as it is manipulated, such as within a beating heart, for example.

In some embodiments, the electroanatomical mapping connector 9 can connect to industry standard systems, for example, allowing the apparatus 12 to be more easily integrated with existing systems. The electroanatomical mapping connector 9 may be connected to any portion of the apparatus 12, such as the elongated flexible conductive element 3, the activator unit 7, the electrosurgical unit connector 8, or the coupler 11, for example. The activator unit 7 also includes a switch element 13, such as a push button or other element, allowing the user to selectively activate the flow of RF energy. The interventional wire 2 is electrically insulated along its length and includes a proximal end portion 16 coupled to the coupler 11 and an uninsulated exposed tip 17 located at a distal end portion 18. As used herein to describe various embodiments from the perspective of a user, “proximal” may refer to a direction generally towards the user of the apparatus, while “distal” may refer to a direction generally away from the user of the apparatus.

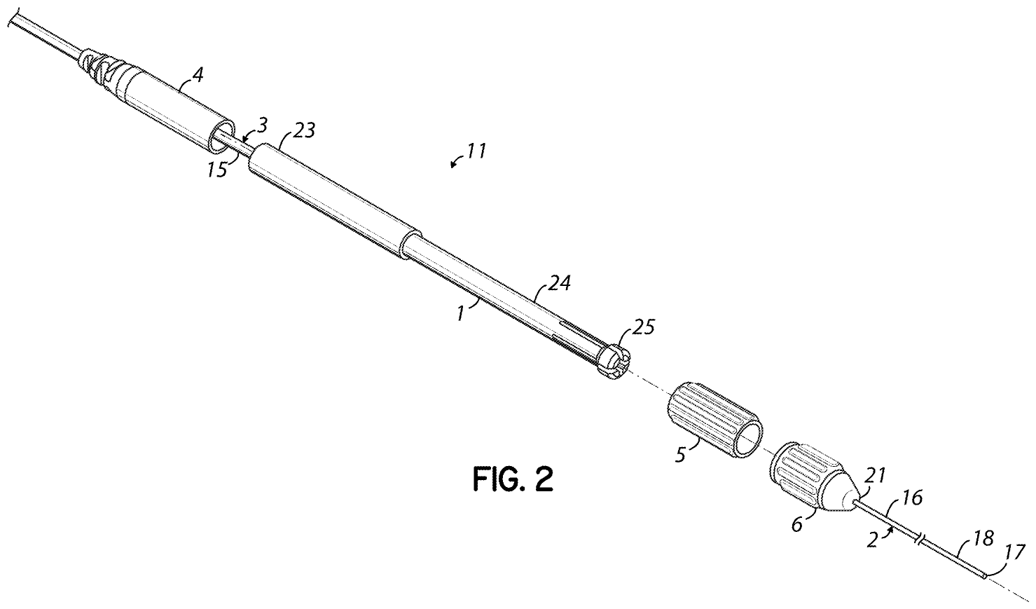

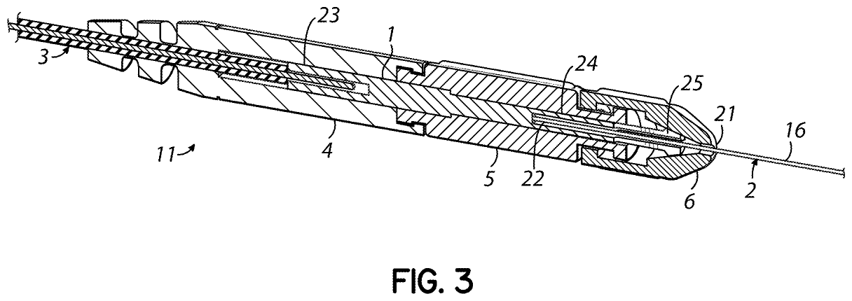

FIG. 2 is an exploded view of the coupler 11 and FIG. 3 is a sectional view of the coupler 11. In this illustrative embodiment, the coupler 11 includes a collet 1, a first housing 4, a second housing 5, and a cap 6. A proximal end portion 23 of the collet 1 is located inside the first housing 4, another portion of the collet 1 is located inside the second housing 5, and a distal end portion 24 of the collet 1 is located inside the cap 6. The second housing 5 is coupled to the first housing 4, and the cap 6 is rotatably coupled to the second housing 5. In some embodiments, the first and second housings 4, 5 are a single component or housing assembly, and the cap 6 is rotatably coupled to a distal end of the housing assembly. In other embodiments, the second housing 5 may be part of the cap 6 and the second housing and cap assembly, or elongated cap, is rotatably coupled to a distal end of the first housing 4. The cap 6 may be tapered at a distal end 21

In this illustrative embodiment, the elongated flexible conductive element 3 is connected, electrically and mechanically, to the interventional wire 2, through the coupler 11. The elongated flexible conductive element 3 is electrically connected to the interventional wire 2, through the collet 1. In this illustrative embodiment, the distal end 15 of the elongated flexible conductive element 3 is connected, electrically and mechanically, to the proximal portion 23 of the collet 1. The proximal end portion 16 of the interventional wire 2 is electrically and mechanically coupled to the distal end portion 24 of the collet 1. In other embodiments, the elongated flexible conductive element 3 may be mechanically coupled to the interventional wire 2 by other means. The proximal end portion 16 of the interventional wire 2 may include an uninsulated proximal end portion 22 to facilitate the electrical connection between the collet 1 and the interventional wire 2. In some embodiments, the collet 1 may include other structures for making the electrical connection with the interventional wire 2. In some embodiments, the coupler 11 may include other structures for making the electrical and/or mechanical connections between the elongated flexible conductive element 3 and the interventional wire 2.

Referring to FIGS. 2 and 3, prior to performing a medical procedure, a medical professional may load the proximal end 16 of the interventional wire 2 into the coupler 11 through the cap 6, for example. When uncompressed or radially expanded, the distal end portion 24 of the collet 1 can receive the proximal end portion 16 of the interventional wire 2. The distal end portion 24 of the collet 1 is then radially compressed or tightened around the proximal end portion 16 of the interventional wire 2 to electrically couple the elongated flexible conductive element 3 to the interventional wire 2. This connection, for example, may be accomplished in a manner where the cap 6 causes movable portions 25 of the distal end portion 24 of the collet 1 to tighten or move radially inward when the cap 6 is rotated in a first direction. Rotating the cap 6 in a second direction causes the movable portions 25 of the collet 1 to expand or move radially outward to loosen the connection to the interventional wire 2. The action of the movable portions 25 of the collet 1 to tighten or compress against the elongated flexible conductive element 3 and the interventional wire 2 releasably connects the elongated flexible conductive element 3 and the interventional wire 2 together. As a result, once the medical procedure, such as transseptal puncture, is performed, the medical professional can disconnect the coupler 11 from the interventional wire 2 to utilize the interventional wire 2 as a guidewire for the delivery of other medical devices.

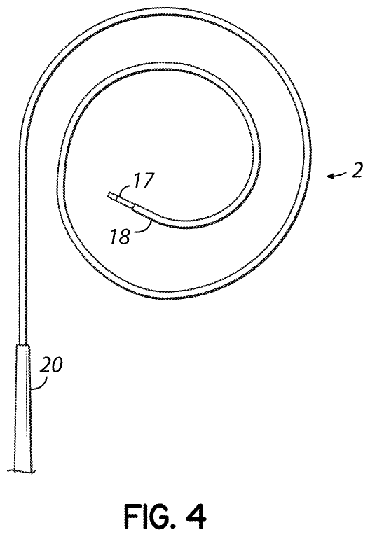

FIG. 4 illustrates the interventional wire 2 passing through an introducer sheath 20 and the exposed tip 17 located at the distal end portion 18 of the interventional wire 2. In this illustrative embodiment, the interventional wire 2 is coated with an electrical insulating material, such as, for example, polytetrafluoroethylene (PTFE). Electrical insulating materials, such as PTFE, serve many functions, such as preventing the loss of RF energy along the length of the interventional wire 2, reducing the friction between the interventional wire 2 and arteries or veins, and preventing the growth of microorganisms. Additionally, the interventional wire 2 contains an exposed tip 17 that enables a controlled discharge of the RF energy from the interventional wire 2 at a surgical site to puncture the septal tissue of the right atrium and access the left atrium.

FIG. 4 also illustrates the interventional wire 2 forming a pigtail, such as would be formed in a patient's heart when a medical professional utilizes this apparatus 12 during a medical procedure, for example. The interventional wire 2 may be, for example, approximately 275 cm long to allow exchange device exchanges while the distal end portion 18 of the interventional wire 2 remains in the left atrium of a patient's heart after a medical procedure, such as a transseptal puncture. In this illustrative embodiment, the interventional wire 2 is approximately 0.032 inches in diameter to be compatible with most transseptal introducer sheaths.

To activate the electrosurgical unit 10 and send RF energy through the electrosurgical unit connector 8, a medical professional will activate the activator unit 7. For example, as shown in FIG. 1, this activation can come in the form of a switch element 13, such as a push button, on the activator unit 7 that, when actively depressed, prompts the flow of RF energy from the electrosurgical unit 10. When the activator unit 7 is activated, RF energy begins to flow through the apparatus 12. The elongated flexible conductive element 3 conducts the RF energy through its proximal end 14, through the mid-portion 19 of the elongated flexible conductive element 3, and subsequently, to the distal end 15 of the elongated flexible conductive element 3 located within the coupler 11. From there, the RF energy is transferred to the collet 1, which conducts the RF energy through the coupler 11 and into the proximal end portion 16 of the interventional wire 2, where the RF energy then travels along the length of the interventional wire 2 to the exposed tip 17 located at the distal end portion 18 of the interventional wire 2, where it is then discharged to perform a puncture, for example. By activating this RF energy, the medical professional can perform a medical procedure such as a transseptal puncture from the right atrium of the heart to the left atrium of the heart. After the interventional wire 2 has been de-energized, and the coupler 11 has been disconnected from the interventional wire 2, the interventional wire 2 can be utilized as a guidewire for the delivery of medical devices.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative product and method and illustrative examples shown and described. Accordingly, departures may be made from such details without departing from the scope of the general inventive concept. For example, any of the individual features or aspects described herein may be utilized alone or together in any combination depending on the desired results and attendant advantages.

What is claimed is:

1. An apparatus for performing a medical procedure comprising:
an electrosurgical unit for generating radiofrequency energy; and
an assembly including:
an elongated flexible conductive element including a proximal end connected to the electrosurgical unit, and a distal end;
an interventional wire configured to deliver radiofrequency energy;
a coupler electrically coupling the distal end of the elongated flexible conductive element to the interventional wire; and
an activator unit electrically coupled to the elongated flexible conductive element and configured to selectively activate the delivery of radiofrequency energy from the electrosurgical unit to the interventional wire.

2. The apparatus of claim 1, wherein the interventional wire comprises:
an insulated wire including a proximal end portion configured for coupling the interventional wire to the coupler, and a distal end portion; and
an uninsulated tip portion located at the distal end portion of the insulated wire and configured to deliver radiofrequency energy.

3. The apparatus of claim 2, wherein the proximal end portion of the interventional wire comprises an uninsulated proximal end portion.

4. The apparatus of claim 1, wherein the elongated flexible conductive element is a cable.

5. The apparatus of claim 1, wherein the coupler comprises a collet configured to electrically couple the elongated flexible conductive element and the interventional wire;
wherein the elongated flexible conductive element is coupled to a proximal end of the collet, and
the interventional wire is coupled to a distal end of the collet.

6. The apparatus of claim 5, wherein the coupler further comprises:
a housing at least partially containing the collet; and
a cap coupled to a distal end of the housing;
wherein the interventional wire is coupled to the collet with the cap.

7. The apparatus of claim 6, wherein the interventional wire is removably coupled to the coupler,
tightening the cap compresses or tightens the distal end of the collet on the interventional wire thereby coupling the interventional wire to the collet, and
loosening the cap allows the distal end of the collet to loosen or expand allowing the interventional wire to be removed from the collet.

8. The apparatus of claim 1, wherein the interventional wire is removably coupled to the coupler.

9. The apparatus of claim 1, wherein the interventional wire serves as a guidewire for the delivery of a medical device.

10. The apparatus of claim 1, wherein the activator unit includes a switch element that is configured to selectively supply radiofrequency energy from the electrosurgical unit through the elongated flexible conductive element to the interventional wire when the switch element is activated.

11. The apparatus of claim 1, wherein the activator unit is spatially separated from the coupler by a portion of the elongated flexible conductive element.

12. The apparatus of claim 1, wherein the activator unit is spatially separated from the proximal end of the elongated flexible conductive element by a segment of the elongated flexible conductive element.

13. The apparatus of claim 1, further comprising an electrosurgical unit connector coupled to the proximal end of the elongated flexible conductive element and configured to connect the elongated flexible conductive element to the electrosurgical unit.

14. The apparatus of claim 1, wherein the elongated flexible conductive element is releasably connected to the electrosurgical unit.

15. The apparatus of claim 1, wherein the apparatus further comprises an electroanatomical mapping connector for connecting the apparatus to an electroanatomical mapping system.

16. An apparatus for performing a medical procedure comprising:
an elongated flexible conductive element including a proximal end configured to connect to an electrosurgical unit for generating radiofrequency energy, and a distal end;
an interventional wire configured to deliver radiofrequency energy;
a coupler electrically coupling the distal end of the elongated flexible conductive element to the interventional wire; and
an activator unit electrically coupled to the elongated flexible conductive element and configured to selectively activate the delivery of radiofrequency energy from the electrosurgical unit to the interventional wire.

17. The apparatus of claim 16, wherein the interventional wire comprises:
an insulated wire including a proximal end portion configured for coupling the interventional wire to the coupler, and a distal end portion; and
an uninsulated tip portion located at the distal end portion of the insulated wire and configured to deliver radiofrequency energy.

18. The apparatus of claim 16, wherein the proximal end portion of the interventional wire comprises an uninsulated proximal end portion.

19. The apparatus of claim 16, wherein the elongated flexible conductive element is a cable.

20. The apparatus of claim 16, wherein the coupler comprises a collet configured to electrically couple the elongated flexible conductive element and the interventional wire;
wherein the elongated flexible conductive element is coupled to a proximal end of the collet, and
the interventional wire is coupled to a distal end of the collet.

21. The apparatus of claim 20, wherein the coupler further comprises:
a housing at least partially containing the collet; and
a cap coupled to a distal end of the housing;
wherein the interventional wire is coupled to the collet with the cap.

22. The apparatus of claim 21, wherein the interventional wire is removably coupled to the coupler,
tightening the cap compresses or tightens the distal end of the collet on the interventional wire thereby coupling the interventional wire to the collet, and
loosening the cap allows the distal end of the collet to loosen or expand allowing the interventional wire to be removed from the collet.

23. The apparatus of claim 16, wherein the interventional wire is removably coupled to the coupler.

24. The apparatus of claim 16, wherein the interventional wire serves as a guidewire for the delivery of a medical device.

25. The apparatus of claim 16, wherein the activator unit includes a switch element that is configured to selectively supply the radiofrequency energy from the electrosurgical unit through the elongated flexible conductive element to the interventional wire when the switch element is activated.

26. The apparatus of claim 16, wherein the activator unit is spatially separated from the coupler by a portion of the elongated flexible conductive element.

27. The apparatus of claim 16, wherein the activator unit is spatially separated from the proximal end of the elongated flexible conductive element by a segment of the elongated flexible conductive element.

28. The apparatus of claim 16, wherein the apparatus further comprises an electroanatomical mapping connector for connecting the apparatus to an electroanatomical mapping system.

29. The apparatus of claim 16, wherein the proximal end of the elongated flexible conductive element includes an electrosurgical unit connector configured to connect the elongated flexible conductive element to an electrosurgical unit.

30. The apparatus of claim 16, wherein the electrosurgical unit connector is configured to couple the elongated flexible conductive element to any one of a plurality of electrosurgical units.

31. A method of performing a medical procedure comprising:
generating radiofrequency energy using an electrosurgical unit;
conducting the radiofrequency energy from the electrical surgical unit through an elongated flexible conductive element to an activator unit;
selectively activating the activator unit to selectively direct the radiofrequency energy to an interventional wire; and
using the interventional wire to deliver the radiofrequency energy to a surgical site.

32. The method of claim 31, wherein the surgical site is the heart and the medical procedure performed is a transeptal puncture.

33. The method of claim 31, wherein the activator is selectively activated by activating and deactivating a switch element.

34. The method of claim 31, further comprising generating an electroanatomical map.

35. The method of claim 31, further comprising guiding the interventional wire to a surgical site.

36. The method of claim 31, further comprising guiding the interventional wire through an introducer sheath.

37. The method of claim 31, further comprising guiding a medical device to a surgical site with the interventional wire.

38. A method of preparing an apparatus for a medical procedure comprising:
mechanically and electrically coupling an elongated flexible conductive element to an electrosurgical unit for conducting radiofrequency energy generated by the electrosurgical unit;
mechanically and electrically coupling the elongated flexible conductive element to a coupler; and
mechanically and electrically coupling an interventional wire to the coupler.

39. The method of claim 38, further comprising coupling the electrosurgical unit to an electroanatomical mapping system.

40. The method of claim 38, further comprising energizing the electrosurgical unit.

41. The method of claim 38, further comprising guiding the interventional wire into an introducer sheath.
